(19) United States
(12) Patent Application Publication
(10) Pub. No.: US 2025/0104814 A1
(43) Pub. Date: Mar. 27, 2025

(54) METHOD AND SYSTEM FOR EVALUATING ENZYMATIC REACTION FEASIBILITY BASED ON MULTIPLE TASKS AND MOLECULAR MULTI-MODAL FEATURES

(71) Applicant: WUHAN UNIVERSITY, Wuhan City (CN)

(72) Inventors: Juan LIU, Wuhan City (CN); Jianghang LIU, Wuhan City (CN); Jing FENG, Wuhan City, Hubei Province (CN)

(73) Assignee: WUHAN UNIVERSITY, Wuhan City (CN)

(21) Appl. No.: 18/662,132

(22) Filed: May 13, 2024

(30) Foreign Application Priority Data
Sep. 26, 2023 (CN) 202311258354.1

(51) Int. Cl. G16B 40/00; G06F 40/242; G06F 40/284

(52) U.S. Cl. CPC G16B 40/00; G06F 40/242; G06F 40/284

(57) ABSTRACT

Provided is a method and a system for evaluating enzymatic reaction feasibility based on multiple tasks and molecular multi-modal features. An enzymatic reaction feasibility dataset is constructed with a public dataset and a bioengineering reaction rule template library; SMILES sequence features and Morgan fingerprint spatial structure features of a product molecule and a substrate molecule of a reaction are used as inputs to a neural network; a dual-branch network is constructed based on an attention mechanism and a convolutional neural network to extract molecular multi-modal features; a product SMILES sequence generation task is taken as a secondary task to strengthen the capability of a model learning sequence feature; richer features are provided for an enzymatic reaction feasibility evaluation task; and the trained model is effectively enabled to accurately determine reaction feasibility by taking the molecular multi-modal features into overall consideration.

### CROSS-REFERENCE TO RELATED APPLICATIONS

This application claims foreign priority benefits under 35 U.S.C. § 119 (a)-(d) to Chinese patent application No. 202311258354.1 filed on Sep. 26, 2023, the disclosure of which is hereby incorporated herein by reference in its entirety.

### TECHNICAL FIELD

The present disclosure belongs to the field of biomolecular synthesis pathway design, particularly relates to a method for evaluating enzymatic reaction feasibility based on multiple tasks and molecular multi-modal features, and more particularly relates to the application of deep learning in the field of biological information.

### BACKGROUND

Nowadays, the synthetic biology applied to the industrial biotechnology is changing the way of producing a biological material, but there are still has many problems needing to be optimized. Bio-retrosynthesis pathway planning is a problem well worthing solution and optimization, this problem specifically refers to, for a complex target molecule, how a reasonable and efficient synthesis route is designed with reference to a tree model structure by using simple and easily available molecules as substrate molecules. The problem of bio-retrosynthesis pathway planning allows for the design of new enzymatic reactions through biometabolic engineering to enable pathways to reach target biomolecules. However, a large number of enzymatic reactions derived from this process result in explosion of various possible combinations. For these combinations, even an experienced biologist can't select a reaction that is most likely to occur, and if an experiment is conducted for verification, a lot of experimental costs may be spent. Therefore, there is a need for a method enabling a computer to automatically screen a large number of enzymatic reactions derived in a retrosynthesis pathway, thereby eliminating a low-feasibility reaction that may be hardly evaluated by human but may be easily identified by the computer and reducing the workload of experts in the biosynthesis field.

Existing methods for evaluating enzymatic reaction feasibility are mainly classified into the following two main categories: one is a category based on biochemical knowledge, by which a field expert determines whether an enzymatic reaction is feasible by considering conditions in the process of a reaction, such as energy changes and entropy changes of products and substrates, chemical bond breaking or formation possibilities, the presence and activity of enzymes, and chassis cell environment; although the determination of the field expert is highly authoritative, this process needs lots of professional knowledge and labor costs. The other one based on machine learning, such methods have achieved good effects on the evaluation of enzymatic reaction feasibility. However, the existing methods do not consider rich sequence features included in a molecular SMILES character string in the model design and merely regard model training as a binary classification task in the model training process. The accuracy and reliability of feasibility evaluation by the model therefore need to be improved.

### SUMMARY

To solve the technical problems described in the above background, the present disclosure provides a method and a system for evaluating enzymatic reaction feasibility based on multiple tasks and molecular multi-modal features. In this method, with reference to features of a plurality of modalities of substrate molecules and product molecules in an enzymatic reaction, a dual-branch feature extraction network based on an attention module mechanism and a convolutional neural network is established, and the training task is expanded to a combination of a product SMILES sequence generation task and a feasibility classification task. The product SMILES sequence generation task enables a model to have stronger sequence extraction capability to assist the feasibility classification task in performing more accurate evaluation. In this method, the problem of enzymatic reaction feasibility is comprehensively considered in terms of molecular sequence features and structure features, the trained model has excellent robustness and adaptability, which can be used to screen out infeasible reactions derived from bio-retrosynthesis and optimize pathway design.

The present disclosure provides the following technical solutions.

In a first aspect, the present disclosure provides a method for evaluating enzymatic reaction feasibility, including the following steps:
S1: collecting a public enzymatic reaction dataset, and forming a positive sample pair dataset by a product molecule and a substrate molecule having a highest similarity matching degree with the product molecule in each enzymatic reaction; obtaining a negative sample pair dataset by expanding with a bioengineering reaction rule template library and the positive sample pair dataset; and randomly mixing the positive sample pair dataset and the negative sample pair dataset in combination with corresponding enzymatic reaction feasibility labels to obtain an enzymatic reaction feasibility dataset D;
S2: calculating multi-modal features consisted of a molecular sequence feature and a molecular spatial structure feature: counting all characters occurring in the dataset D to generate a character dictionary vocab, and converting simplified molecular input line entry system (SMILES) characters of a molecule pair into a digital vector as the molecular sequence feature according to the dictionary vocab and by an Embedding layer; and using open source toolkit RDKit to calculate a Morgan fingerprint of the molecule pair as the molecular spatial structure feature, where the two features provide feature descriptions under different perspectives and are combined to provide more comprehensive and richer molecular feature representations;
S3: establishing a dual-branch feature extraction network based on a convolutional neural network and an attention mechanism network, and using the multi-modal features of molecule pairs in the dataset D as network inputs;
S4: training a model network driven by multiple tasks: the multiple tasks including an enzymatic reaction feasibility evaluation task as a main task and a product SMILES sequence generation task as a secondary task, where the enzymatic reaction feasibility evaluation task is a binary classification task in essence; Based on the idea of machine translation, the product SMILES sequence generation task regards SMILES character changing from the substrate molecule to the product molecule in an enzymatic reaction as a “machine translation” like process; the model network is trained for a plurality of epochs to obtain a Trans-RFC enzymatic reaction feasibility evaluation model; multi-task learning allows different feature extraction modules of the model to share and refer to different modal features of molecules and also enables the model to have stronger generalization capability; the product SMILES sequence generation task allows the model to learn richer and more accurate SMILES sequence feature and transfer the feature to the classification task by sharing an underlying layer parameter of the model, and the performance of the classification task is effectively improved; the model is trained and optimized using a cross-entropy loss function and an Adam algorithm and can be applied to the downstream task;
S5: evaluating enzymatic reaction feasibility using the Trans-RFC enzymatic reaction feasibility evaluation model.

In an implementation, an approach of obtaining and collecting the known feasible enzymatic reaction dataset to obtain the positive sample pair dataset in step S1 may specifically include the following sub-steps:
S1.1: obtaining a MetaNetX public enzymatic reaction dataset;
S1.2: converting a product molecule and a substrate molecules of an enzymatic reaction in the dataset into RDKit molecule objects and calculating similarities, and selecting the substrate molecule having the highest similarity with the product molecule to form a positive sample together with the product molecule, where when a structural similarity of the product molecule and the substrate molecule is higher, a similarity calculation result is closer to 1; in this process, a product molecule and a substrate molecule with a high similarity are selected at a coarse granularity as a molecule pair; and in the positive sample pair dataset formed by all molecule pairs, each sample represents that a corresponding product molecule is obtainable from a substrate molecule in the sample through an enzymatic reaction;
S1.3: installing RetroRules from GitHub, where RetroRules is a toolkit based on bioinformatics and computational chemistry, which may identify a new reaction rule by mining existing biosynthetic reaction and metabolic pathway databases to help to predict potential metabolites and reaction routes; most of new reactions obtained by template prediction, however, are false positive reactions, which is also the reason that the new reactions may be used as negative samples;
S1.4: obtaining the negative sample pair dataset by expanding: calling retrorules-predict function of RetroRules, with an input parameter being the SMILES character string of a substrate molecule in a positive sample and an output result being a set of new reactions with different products generated according to different biochemical reaction rules in RetroRules; randomly selecting one reaction as a negative sample of the substrate molecule, which represents that the reaction is infeasible, and combining the substrate molecule and the product molecule of the reaction into a negative sample pair; and performing the above operations on all substrate molecules in the dataset D to obtain as many negative sample pairs as the positive sample pairs; and
S1.5: randomly mixing the positive and negative sample pair datasets to obtain the enzymatic reaction feasibility dataset D, where each sample includes the SMILES character string of a single substrate molecule, the SMILES character string of a single product molecule, and a corresponding enzymatic reaction feasibility label.

In an implementation, an approach of obtaining molecular features in step S2 may specifically include the following sub-steps:
S2.1: converting molecules SMILES into molecule objects by the open source toolkit RDKit, and calculating the Morgan molecular fingerprints of the molecule objects as structure and property features of the molecules, where a Morgan algorithm is set with a radius parameter r and a number of fingerprint bits fp_dim, and takes stereochemical information into account;
S2.2: counting all characters occurring in the SMILES character strings of all product molecules and substrate molecules in the dataset D as a tokens character set;
S2.3: adding a special character for character embedding to the tokens character set, such as placeholder ‘˜’, start character ‘>’, and end character ‘<’;
S2.4: generating the character dictionary vocab from the tokens character set according to indexes, where the characters are keys and the indexes to the characters are values; for example, the character ‘˜’ is used as a key and the corresponding value is 0, indicating that the value of a digital vector into which the character is converted is 0;
S2.5: modifying the SMILES strings of the molecules in the dataset D according to the following rule: for a substrate molecule, not modifying the SMILES thereof; for a product molecule, the SMILES thereof being a real character sequence that serves as an input to a decoder of a sequence feature extraction module and for comparison with a decoder output, and thus needing to be modified; adding character ‘>’ as a start character to a character head of the SMILES serving as the input to the decoder, and adding character ‘<’ as an end character to a character tail of the SMILES for comparison with the decoder output;
S2.6: performing length padding on the SMILES strings of the molecules in the dataset D to obtain a uniform fixed length ML; if a SMILES length is less than ML, padding character ‘˜’ for a missing part at an end of characters to the length ML; and if a SMILES length exceeds ML, truncating first ML characters to replace the SMILES string to unify dimensions of a subsequent model input; and
S2.7: generating sequence feature vectors of the SMILES character strings of all the molecules in the dataset D according to the dictionary vocab and the Embedding layer to represent sequence features of the molecules.

In an implementation, the dual-branch feature extraction network in step S3 is composed of three modules: a molecular SMILES sequence feature extraction module based on a Transformer network, a molecular structure feature extraction module based on a convolutional neural network and an attention mechanism, and a feature fusion and output module based on a fully connected layer.

The sequence feature extraction module based on Transformer is composed of five parts: an Embedding layer, a character positional encoding layer, an encoder, a decoder, and a max pooling layer, and is configured to fully extract SMILES sequence features in a molecule pair,
where the Embedding layer is configured to map an input discrete digital vector into a dense vector representation to represent information of each character in the SMILES;
the character positional encoding layer is configured to generate a positional encoding vector using sine and cosine functions for adding to a character feature, thereby adding character position information to a sequence;
the encoder is configured to convert an input molecular sequence feature into a high-dimensional feature representation, fully extract molecular multi-modal features using a self-attention layer and a feedforward neural network, and assist the decoder in generating a character sequence;
the decoder is configured to receive an output of the encoder, generate a corresponding product SMILES character sequence by synthesizing the sequence features of a molecule pair using the self-attention layer, an attention layer, and the feedforward neural network, and transfer the sequence features of the molecule pair to a feature fusion and output module; and
the max pooling layer is configured to reduce dimensions of features and extract an important sequence feature therefrom.

The SMILES sequence of a substrate molecule serves as a source sequence in the product SMILES sequence generation task and is subjected to character Embedding and positional encoding, and then the encoder will learn a sequence feature thereof and send an encoding result to the decoder. Similarly, the SMILES of the product molecule serves as a target sequence and is subjected to character Embedding and positional encoding, and then the decoder will learn a sequence feature thereof, and in combination with the encoding information transferred from the encoder, input the sequence features of the molecule pair to output modules of different tasks through different encoder blocks.

A spatial feature extraction module based on a convolutional neural network is composed of a one-dimensional convolutional layer, an attention mechanism layer, and a max pooling layer. The spatial feature extraction module based on a convolutional neural network serves for fulling extracting molecular structure features in a molecule pair.

The feature fusion and output module is composed of a plurality of linear layers; multi-module features input by the sequence feature extraction module and the spatial feature extraction module are taken into account in combination by the feature fusion and output module, and a Relu function is combined to learn a set of weights and bias parameters to adjust importance of different features for an output result and map the result to a predicted scalar value of final 0 to 1, which is finally used in a binary classification task for evaluating the enzymatic reaction feasibility.

In an implementation, a multi-task model optimization strategy in step S4 may specifically include the following sub-steps:
S4.1: the idea of a “machine translation” task is realized with reference to a text generation model in deep learning. The sequence feature extraction module of the model (Trans-RFC enzymatic reaction feasibility evaluation model) outputs a corresponding product SMILES sequence while outputting a sequence feature to the feature fusion and output module, thereby realizing the sequence generation task. By calculating cross entropy loss of the product SMILES sequence and a real product sequence, the model is forced to conclude stronger molecular sequence feature extraction capability, learn richer sequence features at the underlying layer of the model, and transfer the features to the product SMILES sequence generation task and the enzymatic reaction feasibility evaluation task through different decoder blocks of the upper layer of the model to finally assist the enzymatic reaction feasibility evaluation task in making more accurate determination. The multi-class cross entropy loss calculated by the sequence generation task is Loss1.
S4.2: for the enzymatic reaction feasibility evaluation task, the binary cross entropy is used as a loss function, and the enzymatic reaction feasibility is regarded as a binary classification problem. The feature fusion and output module of the model takes the multi-modal features of a substrate molecule and a product molecule into full consideration through the plurality of linear layers, and performs accurate feasibility evaluation on a reaction to be evaluated based on learned knowledge, i.e., performs binary classification on feasibility in essence. The binary cross entropy of an output result and a label value in a real dataset is calculated to obtain a result as Loss2.
S4.3: hyperparameters α and β are set; a weight of the loss function is adjusted, and finally Loss=α*Loss1+β*Loss2, where both of α and β are greater than 0.
S4.4: with Adam as an optimizer and Loss as the loss function, the model is trained on a training set for a certain number of epochs until the binary classification accuracy of the enzymatic reaction feasibility on a validation set tends to be stable, thus obtaining the optimized Trans-RFC enzymatic reaction feasibility evaluation model.

In a possible implementation, step S5 may include the following sub-steps:
using the trained Trans-RFC model to perform feasibility evaluation on a plurality of derived new reactions generated by single-step prediction in a biological synthesis pathway design; with a substrate molecule and a product molecule in each reaction as a molecule pair, calculating the multi-modal features of the molecule pair together as inputs to a prediction function of the Trans-RFC model; screening out an infeasible reaction according to an output result, and reranking the remaining reactions with respect to expansion priority by feasibility.

In a second aspect, the present disclosure provides a system for evaluating enzymatic reaction feasibility based on multiple tasks and molecular multi-modal features, including:
a data obtaining module configured to supplement negative samples for a public feasible enzymatic reaction dataset and convert the public feasible enzymatic reaction dataset into a dataset in the form of molecule pairs in combination with labels as needed by a model, where the molecule pair is composed of a single substrate molecule SMILES and a single product molecule SMILES;
a molecular multi-modal feature extraction module configured to extract multi-modal features of molecules according to the dataset in the form of the molecule pairs in combination with the labels, where the multi-modal features include molecular sequence feature and molecular structure feature;
an enzymatic reaction feasibility evaluation module configured to input the multi-modal features of a molecule pair to a deep learning model, allow the model to further extract and associate features, and output a feasibility evaluation value of a corresponding reaction; and
a multi-task driven model performance optimization module configured to combine an output of the model with a multi-task calculation error loss for feeding back to the model for further parameter optimization, where multiple tasks include a product SMILES sequence generation task and a binary classification task for reaction feasibility.

The present disclosure has following beneficial effects:
(1) The present disclosure uses the multi-modal features of molecules as a basis of determination for enzymatic reaction feasibility. The multi-modal features of a substrate molecule and a product molecule in an enzymatic reaction are taken into overall consideration using the attention mechanism network, the convolutional neural network, and the fully connected layer network, and the product SMILES sequence generation task is used as the secondary task to significantly improve the capability of the model of obtaining sequence feature and assist the model in making more accurate evaluation on the enzymatic reaction feasibility task.
(2) The method provided in the present disclosure may be applied to a bio-retrosynthesis design process. The model will perform feasibility evaluation on a plurality of derived reactions in a prediction result of a single-step model in each retrosynthesis and screen out infeasible reactions; or a plurality of feasible new reactions are reranked, and an expansion order of substrate molecules in bio-retrosynthesis is optimized. The method achieves a good accuracy on both self-owned validation set and test set and can accurately determine whether a sample is positive or negative. The quantity of calculation in the biological synthesis pathway process may be significantly reduced; the experimental verification cost may be reduced; and the efficiency of the bio-retrosynthesis pathway design may be improved.

### BRIEF DESCRIPTION OF THE DRAWINGS

FIG. 1 is a flowchart of obtaining an enzymatic reaction feasibility dataset provided in an embodiment of the present disclosure;

FIG. 2 is a flowchart of obtaining a character dictionary based on a dataset in an embodiment of the present disclosure;

FIG. 3 is an overall structural diagram of a trained network in an embodiment of the present disclosure; and

FIG. 4 is an example application diagram of a Trans-RFC model in the field of biological synthesis pathway designs in an embodiment of the present disclosure.

### DETAILED DESCRIPTION

To make the objectives, technical solutions, and advantages of the embodiments of the present disclosure clearer, the technical solutions in the embodiments of the present disclosure will be described below more clearly and completely with reference to the accompanying drawings in the embodiments of the present disclosure. Apparently, the described embodiments are merely some rather than all of the embodiments of the present disclosure. All other embodiments derived from the embodiments of the present disclosure by those skilled in the art without creative efforts shall fall within the protection scope of the present disclosure.

### Example 1

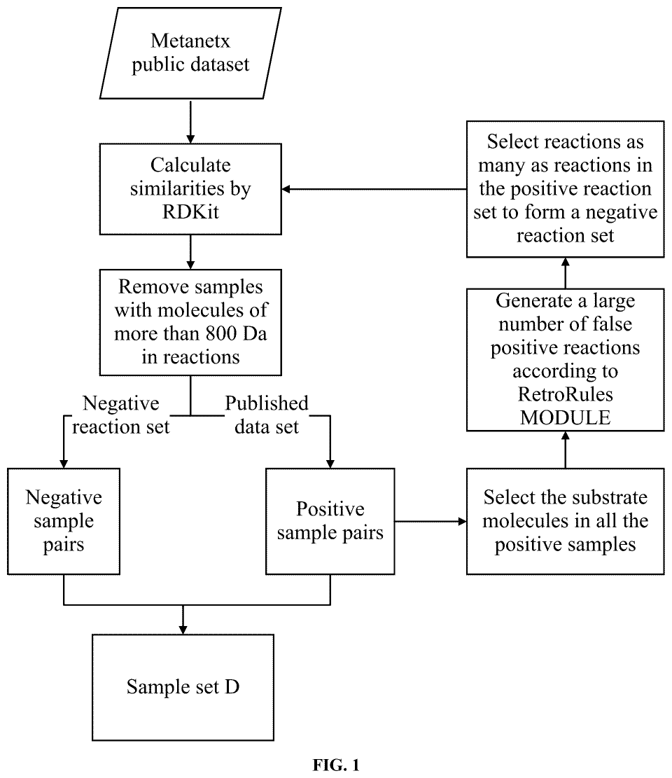

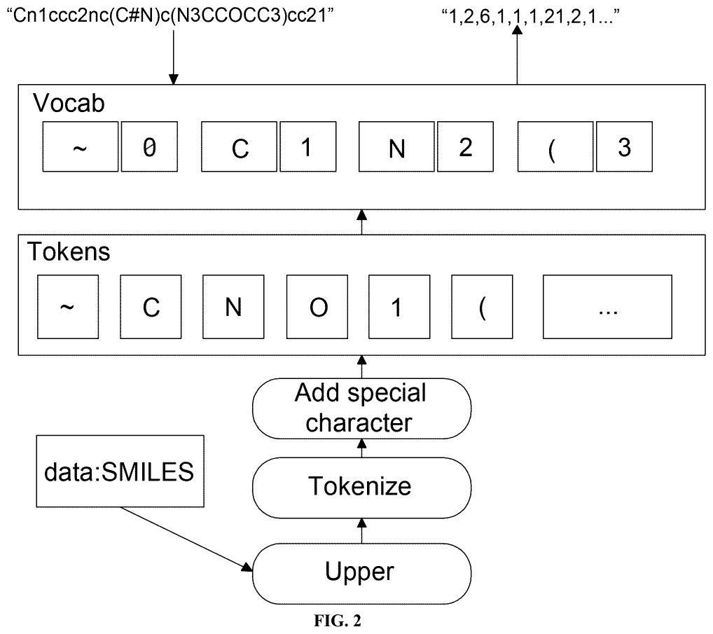

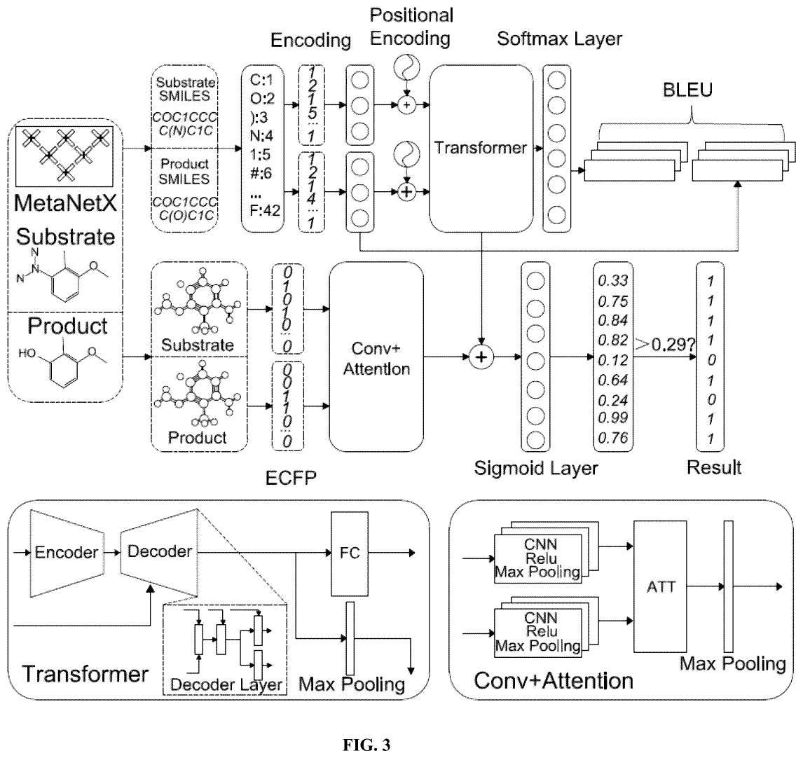

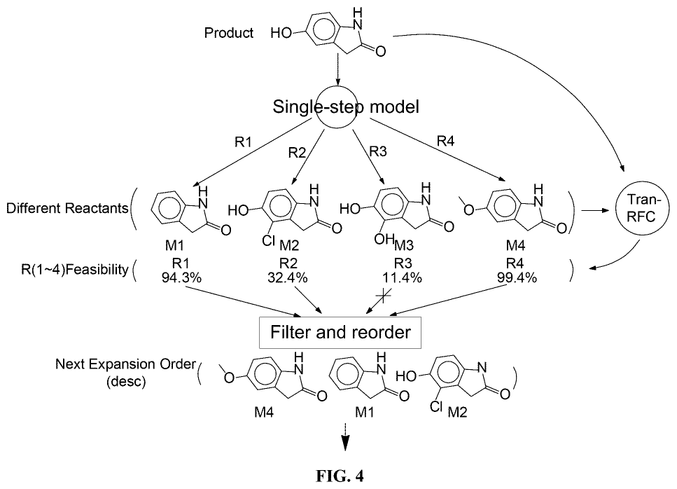

With reference to FIG. 1 which illustrates obtaining an enzymatic reaction feasibility dataset, FIG. 2 which illustrates a flowchart of obtaining a character dictionary, FIG. 3 which illustrates an overall structure of a model, and FIG. 4 which illustrates an example application of Trans-RFC model in the field of biological synthesis, the present disclosure provides the following technical solution: a method for evaluating enzymatic reaction feasibility based on multiple tasks and molecular multi-modal features includes the following steps.

S1: a public enzymatic reaction dataset is collected, and a product molecule and a substrate molecule having the highest similarity matching degree with the product molecule in each enzymatic reaction together form a positive sample pair dataset; a negative sample pair dataset is obtained by expanding with a bioengineering reaction rule template library and the positive sample pair dataset; and the positive sample pair dataset and the negative sample pair dataset are randomly mixed in combination with corresponding enzymatic reaction feasibility labels to obtain an enzymatic reaction feasibility dataset D.

S2: multi-modal features consist of molecular sequence feature and molecular spatial structure feature are calculated: all characters occurring in the dataset D are counted to generate a character dictionary vocab, and SMILES character features of a molecule pair are converted into a digital vector as the molecular sequence feature according to the dictionary vocab and by an Embedding layer; and open source toolkit RDKit is used to calculate a Morgan fingerprint (i.e., extended-connectivity fingerprint (ECFP)) of the molecule pair as the molecular spatial structure feature, where the two features provide feature descriptions under different perspectives and are combined to provide more comprehensive and richer molecular representations.

S3: a dual-branch feature extraction network based on a convolutional neural network and an attention mechanism network is established, and the multi-modal features of molecule pairs in the dataset D are used as network inputs.

S4: a model network is trained driven by multiple tasks: the multiple tasks includes an enzymatic reaction feasibility evaluation task as a main task and a product SMILES sequence generation task as a secondary task, where the enzymatic reaction feasibility evaluation task is a binary classification task in essence; the product SMILES sequence generation task regards, based on the machine translation idea, SMILES character changing from the substrate molecule to the product molecule in the enzymatic reaction as a “machine translation” like process; and the model network is trained for a plurality of epochs to obtain a Trans-RFC enzymatic reaction feasibility evaluation model. Multi-task learning allows different feature extraction modules of the model to share and refer to different modal features of molecules and also enables the model to have stronger generalization capability. The sequence generation task allows the model to learn richer and more accurate SMILES sequence features and transfer the features to the classification task by sharing an underlying layer parameter of the model, and the performance of the classification task is effectively improved; and the model is trained and optimized using the cross entropy loss function and Adam algorithm and may be applied to a downstream task.

S5: reaction feasibility is evaluated using the Trans-RFC enzymatic reaction feasibility evaluation model.

The technical solution of the embodiment of the present disclosure is described below with reference to FIG. 1 to FIG. 4.

In an implementation, an approach of obtaining and collecting the known feasible enzymatic reaction dataset (positive sample set) in step S1 specifically includes the following sub-steps.

S1.1: a MetaNetX public enzymatic reaction dataset is obtained from MetaNetX official website, where the public MetaNetX enzymatic reaction dataset contains 62369 existing enzymatic reactions; the reaction of a single data is composed of a single product molecule SMILES and a single one or more substrate molecules SMILES, which are joined by character string “>>” to form a complete enzymatic reaction.

S1.2: the molecules SMILES are converted into RDKit molecule objects and similarities are calculated by MolFromSmiles function in the open source toolkit RDKit, and the substrate molecule having the highest similarity with the product molecule is selected to form a positive sample molecule pair together with the product molecule, where when the structural similarity of the product molecule and the substrate molecule is higher, the similarity calculation result is closer to 1. In this process, a product molecule and a substrate molecule with the high similarity are selected at a coarse granularity as a molecule pair; and in the positive sample pair dataset formed by all molecule pairs in combination with labels, each sample represents that the corresponding product molecule is obtainable from the substrate molecule in the sample through an enzymatic reaction;

To reduce the calculation complexity and to make the molecular structure feature more representative to satisfy experimental feasibility, some samples of which the product molecules or substrate molecules have a molecular weight of more than 800 Da are eliminated.

S1.3: RetroRules toolkit is instralled from RetroRules official website or GitHub. RetroRules biochemical reaction template library is used. RetroRules is a toolkit based on bioinformatics and computational chemistry, which may identify a new reaction rule by mining existing biosynthetic reaction and metabolic pathway databases to help to predict potential metabolites and reaction routes; most of new reactions obtained by template prediction, however, are false positive reactions, which is also the reason that the new reactions may be used as negative samples.

S1.4: the negative sample pair dataset is obtained by expanding: retrorules-predict function in the toolkit is called; an input parameter is the SMILES character string of a substrate molecule in a positive sample and an output result is a set of new reactions with different products generated according to different biochemical reaction rules; one reaction is randomly selected as a negative sample of the substrate molecule, and the substrate molecule and the product molecule of the reaction form a negative sample pair; and the above operations are performed on all substrate molecules to obtain as many negative sample pairs as the positive sample pairs.

S1.5: the positive and negative sample pairs are randomly mixed to form the enzymatic reaction feasibility dataset D, and 75000 pieces of data with labels are randomly selected therefrom as a final dataset, where each piece of data is composed of a substrate molecule SMILES, a product molecule SMILES, and a corresponding enzymatic reaction feasibility label; the label being 1 represents a positive sample; and the label being 0 represents a negative sample.

FIG. 1 is a flowchart of establishing the dataset D in combination with S1.

In an implementation, an approach of obtaining molecular features in step S2 specifically includes the following sub-steps

S2.1: structure features of molecules are obtained: the following operations are performed on all molecules in the dataset D: a molecule SMILES is converted into a molecule object using the open source toolkit RDKit, and the Morgan fingerprint of the molecule object is calculated and converted into a digital vector that represents the spatial structure and property feature of the molecule, where a Morgan algorithm is set with a radius parameter 2 and a number of fingerprint bits 2048, and set to take chiral (stereochemical) information into account.

S2.2: all molecules SMILES in the dataset D are unified as English capital letters, and all characters occurring in the SMILES character strings of all molecules in the dataset D are counted, defined as a tokens character set. By statistics, there are a total of 39 types of different characters, and typical characters are ‘C’, ‘N’, ‘O’, and the like.

S2.3: three special characters, i.e., placeholder ‘˜’, start character ‘>’, and end character ‘<’, for character embedding are added to the tokens character set, after which there are a total of 42 types of different characters in the tokens character set.

S2.4: generating the character dictionary vocab from the tokens character set according to indexes, where the characters are keys and the indexes to the characters are values. For example, the value of character ‘˜’ as an index is 0, indicating that the value of a digital vector into which the character is converted is 0. Each character corresponds to a unique index value, and a size of vocab is 42.

S2.5: the SMILES strings of the molecules in the dataset D are modified according to the following rule: for a substrate molecule, the SMILES thereof is not modified; for a product molecule, the SMILES thereof is a real character sequence that serves as an input to a decoder of a sequence feature extraction module and for comparison with a decoder output, and thus needs to be modified. Character ‘>’ is added as a start character to a character head of the SMILES serving as the input to the decoder, and character ‘<’ is added as an end character to a character tail of the SMILES for comparison with the decoder output. A formula of the rule for modifying SMILES is as follows:

$$new\_SMILES = \{ \begin{matrix}SMILES & SMILES ∈ {U}_{substrate} \\ {}^{′} {>}^{′} + SMILES \vert SMILES + {}^{′} {<}^{′} & SMILES ∈ {U}_{product}\end{matrix}$$

S2.6: all molecular SMILES strings in the dataset D are padded. By statistics, about 83% of SMILES strings in the dataset have a length of less than 120. Since an excessively long SMILES may not be representative of biomolecules, a uniform fixed length is 120, and the molecules SMILES not meeting the condition are padded according to the following rule: if a SMILES length is less than 120, character ‘˜’ is padded for a missing part at an end of characters to the length of 120; and if a SMILES length exceeds 120, first 120 characters are truncated to replace the SMILES string to unify dimensions of a subsequent model input. A formula of the rule for modifying the SMILES is as follows:

$$new\_SMILES = \{ \begin{matrix}sub ( SMILES , 0 , 120 ) & len ( SMILES ) > 120 \\ ljust ( SMILES , 120 , {}^{′} {∼}^{′} ) & len ( SMILES ) <= 120\end{matrix}$$

S2.7: a molecular sequence feature vector is generated from each character in the modified SMILES character strings of all the molecules in the dataset D according to the dictionary vocab, which represents the sequence feature of the molecule, where the size of the vocab is 42.

FIG. 2 is a flowchart of establishing the vocab in combination with S2.

In an implementation, the dual-branch feature extraction network based on a convolutional neural network and an attention mechanism network in step S3 is composed of three modules: a molecular SMILES sequence feature extraction module based on a Transformer network, a molecular structure feature extraction module based on a convolutional neural network and an attention mechanism, and a feature fusion and output module based on a fully connected layer.

The sequence feature extraction module based on Transformer is composed of five parts: an Embedding layer, a character positional encoding layer, an encoder layer, a decoder layer, and a max pooling layer, and is configured to fully extract SMILES sequence features in a molecule pair,
where the Embedding layer is configured to map an input discrete digital vector into a dense vector representation to represent information of each character in the SMILES; and hidden unit dimensions d_{model} are set to 512 dimensions.

A formula for positional encoding is as follows:

$${PE}_{pos , 2 i} = sin ( pos / {10000}^{2 i / {d}_{model}} )$$

$${PE}_{pos , 2 i + 1} = cos ( pos / {10000}^{2 i / {d}_{model}} )$$

where pos represents a position index in a sequence; i represents a dimension index; d_{model} represents hidden unit dimensions of each character after Embedding of the sequence.

The character positional encoding layer is configured to generate a positional encoding vector using sine and cosine functions for adding to a character feature, thereby adding character position information to a sequence.

The encoder is configured to convert an input molecular sequence feature into a high-dimensional feature representation, fully extract molecular multi-modal features using a self-attention layer and a feedforward neural network, and assist the decoder in generating a character sequence.

The decoder is configured to receive an output of the encoder, generate a corresponding product SMILES character sequence by synthesizing the sequence features of a molecule pair using the self-attention layer, an attention layer, and the feedforward neural network, and transfer the sequence features of the molecule pair to a feature fusion and output module.

The max pooling layer is configured to reduce dimensions of features and extract an important sequence feature therefrom.

To realize sharing of a SMILES sequence feature, three encoder blocks and two decoder blocks serve as a shared network for a plurality of tasks. To allow for better outputs from different tasks, after the network is shared, a separate decoder block is used for a plurality of tasks to output the sequence feature to realize fine adjustment of an upper layer parameter of the model. In each block of the encoder and the decoder, the molecular sequence feature is fully extracted using a multi-head attention mechanism, a residual, and a feedforward connection network, where the value of Q, K, and V keys of the attention module is 64, and a number of heads is 8. The encoder uses a padding mask to block out useless padded information in encoding, and the shared decoder block uses both of a padding mask and a future mask to block out useless information and information from the future in decoding.

For the product SMILES sequence generation task, the decoder block for the task will output a sequence feature vector of 64*120*512 dimensions for sequence feature comparison, where 64 is batch_size; 120 is a length of the SMILES string; and 512 is the hidden unit dimensions of each character in the SMILES. For the enzymatic reaction feasibility classification task, the decoder block for the task will pool a sequence feature to 512 dimensions on the hidden dimensions and then outputs the pooled sequence feature to the feature fusion and output module.

The SMILES sequence of a substrate molecule serves as a source sequence in the sequence generation task and is subjected to character Embedding and positional encoding, and then the encoder will learn a sequence feature thereof and send an encoding result to the decoder. Similarly, the SMILES of the product molecule serves as a target sequence and is subjected to character Embedding and positional encoding, and then the decoder will also learn a sequence feature thereof, and in combination with the encoded feature transferred from the encoder, input the sequence features of the molecule pair to output modules of different tasks through different encoder blocks.

A spatial feature extraction module based on a convolutional neural network is composed of a one-dimensional convolutional module, an attention layer, and a max pooling layer.

The one-dimensional convolutional block is configured to fully extract spatial features of different scales in the fingerprint features of a product molecule and a substrate molecule, map a sparse spatial feature vector arrangement into a dense arrangement by a sliding window, allowing for richer features, and input a result to the attention layer. The module includes a plurality of convolutional layers, Relu function, and a pooling layer. In a specific implementation, the dimensions of a single molecular fingerprint are 2048, and after passing through the one-dimensional convolutional layer module, change to 64*1024, 64*512, 256*256, and 512*128.

The attention layer is configured to receive the spatial structure features from the product molecule and the substrate molecule, cause each element in the spatial feature sequences of the product molecule and the substrate molecule to thoroughly learn information associated with each element of the opponent by the attention mechanism, and allow a sub-structure feature of the spatial feature sequence to obtain a long distance dependency with a sub-structure feature of the opponent by a global attention mechanism. In a specific implementation, the hidden dimensions input to the attention mechanism are 128, and a key value vector is 64, and the dimensions of a structure after passing through the attention layer are 512*128.

The max pooling layer is configured to further reduce dimensions of spatial features captured by the attention layer and extract the most important spatial structure feature, thereby facilitating the reduction of the calculation complexity. After passing through the max pooling layer, the spatial feature of each molecule is down-sampled to 512.

The feature fusion and output module based on a fully connected layer is composed of a plurality of linear layers; multi-module features input by the sequence feature extraction module and a spatial feature extraction module are taken into account in combination by the feature fusion and output module, and a Relu function in the module learns a set of weight and bias parameters to adjust importances of different features for an output result and maps the result to a predicted scalar value of final 0 to 1, which is finally used in a binary classification task for evaluating the enzymatic reaction feasibility. The closer to 1 the result, the higher the reaction feasibility.

In an implementation, a multi-task model optimization strategy in step S4 includes the following sub-steps.

S4.1: the idea of a “machine translation” task is realized with reference to a text generation model in deep learning. The sequence feature extraction module of the model (Trans-RFC enzymatic reaction feasibility evaluation model) outputs a corresponding product SMILES sequence while outputting a sequence feature to the feature fusion and output module, thereby realizing the sequence generation task. By calculating the cross entropy loss of the product SMILES sequence and the real product sequence in a sample, the model is forced to have stronger capability of extracting molecular sequence features, learn richer and more accurate sequence features at the underlying layer of the model, and transfer the features to the product SMILES sequence generation task and the reaction feasibility evaluation task through different decoder blocks of the upper layer of the model to finally assist the reaction feasibility evaluation task in making more accurate determination. The multi-class cross entropy loss calculated by the sequence generation task is Loss1.

A formula for calculating the multi-class cross entropy loss is as follows:

$${Loss}_{1} = - {\sum }_{i = 1}^{n} {y}_{i} log ( p ( {x}_{i} ) )$$

where y represents a real label, and p (x) represents a model output.

S4.2: for a binary classification task for enzymatic reaction feasibility realized by Trans-RFC whole network, the binary cross entropy is used as a loss function, and the enzymatic reaction feasibility is regarded as a binary classification problem. The feature fusion and output module of the model takes the multi-modal features of a substrate molecule and a product molecule into full consideration through the plurality of linear layers, and performs accurate feasibility evaluation on a reaction to be evaluated based on learned knowledge, i.e., performs binary classification determination on feasibility in essence. The binary cross entropy of an output result and a label value in a real data is calculated to obtain a result as Loss2.

A formula for calculating the binary cross entropy loss is as follows:

$${Loss}_{2} = - [ y log p ( x ) + ( 1 - y ) log ( 1 - p ( x ) ) ]$$

where y represents a real label, and p (x) represents a model output.

S4.3: hyperparameters are set as α=2 and β=1, and a synthetic Loss is an addition sum of α*Loss1 and β*Loss2.

Finally, a formula for calculating Loss is as follows:

$$Loss = α * {Loss}_{1} + β * {Loss}_{2}$$

$$where α = 2 , and β = 1.$$

S4.4: with Adam as an optimizer and Loss as the loss function, the model is trained on a training set for a certain number of epochs until the binary classification accuracy of the enzymatic reaction feasibility on a validation set tends to be stable, thus obtaining the optimized Trans-RFC enzymatic reaction feasibility evaluation model.

In the training process, the synthetic Loss as the loss function and Adam gradient descent algorithm are used to look for an optimal model. In this implementation, the model is trained for 35 epochs at an initial learning rate of 0.0003 with a value of batch_size of 64 until the prediction accuracy of the model on the validation set reaches convergence.

FIG. 3 is an overall structural diagram of a network trained in combination with S3.

In a specific embodiment, step S5 includes the following sub-steps:
use the trained Trans-RFC model to perform feasibility evaluation on a plurality of derived new reactions generated by single-step prediction in a biological synthesis pathway design; with a substrate molecule and a product molecule in each reaction as a molecule pair, calculate the multi-modal features of the molecule pair together as inputs to a prediction function of the Trans-RFC model; screen out an infeasible reaction according to an output result, and rerank the remaining reactions with respect to expansion priority by feasibility.

To verify the effectiveness and feasibility of the method of the present disclosure, validation is performed on the self-owned test value in this embodiment. The model network is trained by the method provided in the present disclosure, and the optimized Trans-RFC model is tested with respect to performance on the test set. The used test set is derived from the self-owned dataset as constructed above. Since the model output is mapped to be between 0 and 1, after testing of a plurality of continuous sets of different thresholds, the enzymatic reaction feasibility threshold is selected as 0.29, and the feasibility determination accuracy of the method on the test set is 92.3%, which has a significant increase as compared with the accuracy of 82.5% of a previous method based on deep learning on the test set. This indicates that the method provided in the present disclosure is effective.

FIG. 4 illustrates an application example of the Trans-RFC model in a biological synthesis pathway design. The method of the present disclosure effectively screens the prediction results of the single-step model and adjusts an order thereof, making a multi-step design in the expansion process of a synthetic search tree more reasonable.

### Example 2

A system for evaluating enzymatic reaction feasibility based on multiple tasks and molecular multi-modal features includes:
a data obtaining module configured to supplement negative samples for a public feasible enzymatic reaction dataset and convert the public feasible enzymatic reaction dataset into a dataset in the form of molecule pairs in combination with labels as needed by a model, where the molecule pair is composed of a single substrate molecule SMILES and a single product molecule SMILES;
a molecular multi-modal feature extraction module configured to extract multi-modal features of molecules according to the dataset in the form of the molecule pairs in combination with the labels, where the multi-modal features include molecular sequence feature and structure feature;
an enzymatic reaction feasibility evaluation module configured to input the multi-modal features of a molecule pair to a deep learning model, allow the model to further extract and associate features, and output a feasibility evaluation value of a corresponding reaction; and
a multi-task driven model performance optimization module configured to combine an output of the model with a multi-task calculation loss for feeding back to the model for further parameter optimization, where multiple tasks include a product SMILES sequence generation task and a binary classification task for reaction feasibility.

The foregoing embodiments are only used to explain the technical solutions of the present disclosure, and are not intended to limit the same. Although the present disclosure is described in detail with reference to the foregoing embodiments, those of ordinary skill in the art should understand that they can still modify the technical solutions described in the foregoing embodiments, or perform equivalent substitutions on some technical features therein. These modifications or substitutions do not make the essence of the corresponding technical solutions deviate from the spirit and scope of the technical solutions of the embodiments of the present disclosure.

The foregoing are merely descriptions of the specific embodiments of the present disclosure, and the protection scope of the present disclosure is not limited thereto. Any modification, equivalent replacement, improvement, etc. made within the technical scope of the present disclosure by those skilled in the art shall be included within the protection scope of the present disclosure.

What is claimed is:

1. A method for evaluating enzymatic reaction feasibility based on multiple tasks and molecular multi-modal features, comprising the following steps:
S1: collecting a public enzymatic reaction dataset, and forming a positive sample pair dataset by a product molecule and a substrate molecule having a highest similarity matching degree with the product molecule in each enzymatic reaction; obtaining a negative sample pair dataset by expanding with a bioengineering reaction rule template library and the positive sample pair dataset; and randomly mixing the positive sample pair dataset and the negative sample pair dataset in combination with sample labels to obtain an enzymatic reaction feasibility dataset D;
S2: calculating molecular multi-modal features formed by combining a molecular sequence feature and a molecular spatial structure feature: counting all characters occurring in the dataset D to generate a character dictionary vocab, and converting a simplified molecular input line entry system (SMILES) character sequence of a molecule pair into a digital vector as the molecular sequence feature according to the dictionary vocab and an Embedding layer; and using an open source toolkit RDKit to calculate a Morgan fingerprint of the molecule pair as the molecular spatial structure feature;
S3: establishing a dual-branch feature extraction network based on a convolutional neural network and an attention mechanism network, and using the multi-modal features of molecule pairs in the dataset D as network inputs;
S4: training a model network driven by multiple tasks: the multiple tasks comprising an enzymatic reaction feasibility evaluation task as a main task and a product SMILES sequence generation task as a secondary task, wherein the enzymatic reaction feasibility evaluation task is a binary classification task; the product SMILES sequence generation task regards SMILES character changing from the substrate molecule to the product molecule in the enzymatic reaction as a “machine translation” like process; and the model network is trained for a plurality of epochs to obtain a Trans-RFC enzymatic reaction feasibility evaluation model; and
S5: evaluating enzymatic reaction feasibility using the Trans-RFC enzymatic reaction feasibility evaluation model.

2. The method for evaluating enzymatic reaction feasibility based on multiple tasks and molecular multi-modal features according to claim 1, wherein step S1 comprises the following sub-steps:
S1.1: collecting the public enzymatic reaction dataset as original data of a positive sample pair dataset, wherein each reaction is composed of a single product molecule and a single one or more substrate molecules; and the product molecule and the substrate molecule are represented by SMILES character strings, respectively;
S1.2: using the open source toolkit RDKit to obtain similarities of a product molecule with a corresponding plurality of substrate molecules: converting the product molecule and the substrate molecules in an enzymatic reaction into RDKit molecule objects and calculating similarities, and selecting the substrate molecule having the highest similarity with the product molecule to form a positive sample together with the product molecule, wherein when a structural similarity of the product molecule and the substrate molecule is higher, a similarity calculation result is closer to 1; the selected product molecule and substrate molecule with a high similarity serve as a single molecule pair; all molecule pairs form the positive sample pair dataset; and the data of each sample represents that a corresponding product is obtainable from a substrate in the sample through an enzymatic reaction;
S1.3: installing RetroRules from GitHub;
S1.4: obtaining the negative sample pair dataset by expanding: calling retrorules-predict function in a RetroRules toolkit, with an input parameter being the SMILES character string of a substrate molecule in a positive sample and an output result being a set of new reactions with different products generated according to different biochemical reaction templates in a RetroRules reaction rule library; randomly selecting one generated new reaction as a negative sample of the substrate molecule, and combining a product molecule of the reaction with a corresponding substrate molecule according to step 1 as a negative sample pair; and performing the above operations on each substrate molecule in the dataset D to obtain the negative sample pair dataset having as many sample pairs as the positive sample pairs; and
S1.5: randomly mixing the positive sample pair dataset and the negative sample pair dataset to obtain the enzymatic reaction feasibility dataset D.

3. The method for evaluating enzymatic reaction feasibility based on multiple tasks and molecular multi-modal features according to claim 2, wherein in step S1.5, the enzymatic reaction feasibility dataset D is formed by randomly mixing positive and negative sample pairs in combination with labels, wherein each piece of data is composed of the SMILES character string of a single substrate molecule, the SMILES character string of a single product molecule, and a corresponding enzymatic reaction feasibility label; the label being 1 represents that the sample is a positive sample; and the label being 0 represents that the sample is a negative sample.

4. The method for evaluating enzymatic reaction feasibility based on multiple tasks and molecular multi-modal features according to claim 1, wherein step S2 comprises the following sub-steps:
S2.1: converting molecules SMILES into molecule objects by the open source toolkit RDKit, and calculating the Morgan fingerprints of the molecule objects as structure and property features of the molecules, wherein a Morgan algorithm is set with a radius parameter r and a number of fingerprint bits fp_dim, and takes stereochemical information into account;
S2.2: counting characters occurring in the SMILES character strings of all molecules in the dataset D as a tokens character set;
S2.3: adding a special character for character embedding to the tokens character set;
S2.4: generating the character dictionary vocab from the tokens character set according to indexes to characters, wherein the characters are keys and the indexes to the characters are values;
S2.5: modifying the SMILES strings of the molecules in the dataset D according to the following rule: for a substrate molecule, not modifying the SMILES thereof; for a product molecule, adding character ‘>’ as a start character to a character head of the SMILES serving as an input to a decoder, and adding character ‘<’ as an end character to a character tail of the SMILES for comparison with a decoder output;
S2.6: performing length padding on the SMILES strings of the molecules in the dataset D to obtain a uniform fixed length ML; and
S2.7: generating sequence feature vectors of the SMILES character strings of all the molecules in the dataset D according to the dictionary vocab and the Embedding layer to represent sequence features of the molecules.

5. The method for evaluating enzymatic reaction feasibility based on multiple tasks and molecular multi-modal features according to claim 4, wherein in step 2.6, an approach of performing length padding on the SMILES strings of the molecules in the dataset D to obtain a uniform fixed length ML comprises: if a SMILES length is less than ML, padding character ‘˜’ for a missing part at an end of characters to the length ML; and if a SMILES length exceeds ML, truncating first ML characters to replace the SMILES string to unify dimensions of a subsequent model input while maximizing the molecular sequence feature.

6. The method for evaluating enzymatic reaction feasibility based on multiple tasks and molecular multi-modal features according to claim 1, wherein the dual-branch feature extraction network in step S3 is composed of three modules: a molecular SMILES sequence feature extraction module based on a Transformer network, a molecular structure feature extraction module based on a convolutional neural network and an attention mechanism, and a feature fusion and output module based on a fully connected layer,
wherein:
the molecular SMILES sequence feature extraction module based on a Transformer network is composed of five parts: an Embedding layer, a character positional encoding layer, an encoder, a decoder, and a max pooling layer,
wherein the Embedding layer is configured to map an input discrete digital vector into a dense vector representation to represent information of each character in a molecule SMILES;
the character positional encoding layer is configured to generate a positional encoding vector using sine and cosine functions for adding to a character feature, thereby adding character position information to a sequence;
the encoder is configured to convert an input molecular sequence feature into a high-dimensional feature representation, fully extract molecular multi-modal features using a self-attention layer and a feedforward neural network, and assist the decoder in generating a character sequence;
the decoder is configured to receive an output of the encoder, generate a corresponding product SMILES character sequence by synthesizing the sequence features of a molecule pair using the self-attention layer, an attention layer, and the feedforward neural network, and transfer the sequence features of the molecule pair to a feature fusion and output module;
the max pooling layer is configured to reduce dimensions of features and extract an important sequence feature therefrom;
the molecular structure feature extraction module based on a convolutional neural network is composed of a one-dimensional convolutional module, an attention layer, and a max pooling layer, wherein the one-dimensional convolutional module is configured to fully extract spatial features of different scales in a molecular fingerprint, and map a sparse spatial feature vector arrangement into a dense arrangement by a sliding window of a convolutional layer, allowing for richer features; the attention layer is configured to cause each element in structure feature sequences of a product molecule and a substrate molecule to thoroughly learn information associated with each element of the opponent and allow a sub-structure feature of the structure feature sequence to obtain a long distance dependency with a sub-structure feature of the opponent by global attention; and the max pooling layer is configured to reduce dimensions of structure features captured by the attention layer and extract an important structure feature from the features as an output;
the feature fusion and output module based on a fully connected layer is composed of a plurality of linear layers; multi-module features input by the sequence feature extraction module and a spatial feature extraction module are taken into account in combination by the feature fusion and output module, and a Relu function in the module learns a set of weight and bias parameters to adjust importances of different features for an output result and maps the result to a predicted scalar value of 0 to 1, which is used in a binary classification task for evaluating the enzymatic reaction feasibility.

7. The method for evaluating enzymatic reaction feasibility based on multiple tasks and molecular multi-modal features according to claim 6, wherein in step S3, the molecular SMILES sequence feature extraction module based on a Transformer network comprises three encoder blocks and two decoder blocks as a shared network for multiple tasks to realize sharing of a sequence feature in multiple tasks, and after the network is shared, a separate decoder block is used for multiple tasks to output the sequence feature to realize fine adjustment of an upper layer parameter of the model; in each block of the encoder and the decoder, the molecular sequence feature is fully extracted using a multi-head attention mechanism, a residual, and a feedforward connection network; and wherein the encoder uses a padding mask to block out padded useless information in encoding, and the decoder uses a padding mask and a future mask to block out padded useless information and information from the future in decoding.

8. The method for evaluating enzymatic reaction feasibility based on multiple tasks and molecular multi-modal features according to claim 7, wherein step S4 comprises the following sub-steps:
S4.1: after sequence features of a molecule pair pass through the shared network, transferring the sequence features to the product SMILES sequence generation task and the enzymatic reaction feasibility evaluation task via different decoder blocks of the upper layer of the Trans-RFC model; and in the product SMILES sequence generation task, calculating, by the model, a multi-class cross entropy loss of a generated product sequence and a real product sequence as Loss1 according to the sequence features;
S4.2: multi-modal features of the molecule pair being to pass through a plurality of fully connected layers of the feature fusion and output module of the Trans-RFC model, performing accurate feasibility evaluation on a reaction to be evaluated, calculating a binary cross entropy of an output result and a real label value in the dataset, and taking a calculated loss as Loss2;
S4.3: setting hyperparameters α and β, adjusting a weight of a loss function, and finally Loss=α*Loss1+β*Loss2, wherein both of α and β are greater than 0; and
S4.4: with Adam as an optimizer and Loss as the loss function, training the model on a training set for a certain number of epochs until a prediction accuracy of a reaction feasibility classification task on a validation set tends to be stable, thus obtaining the optimized Trans-RFC enzymatic reaction feasibility evaluation model.

9. The method for evaluating enzymatic reaction feasibility based on multiple tasks and molecular multi-modal features according to claim 1, wherein step S5 comprises the following sub-steps: calculating the multi-modal features of a substrate molecule and a product molecule of an enzymatic reaction in need of feasibility evaluation according to S2; inputting the multi-modal features to the Trans-RFC model to obtain a feasibility evaluation value of the reaction, wherein when the evaluation value is closer to 1, the reaction feasibility is higher; and comparing an evaluation result with a reference threshold obtained based on data during training to evaluate the final feasibility of the reaction.

10. A system for evaluating enzymatic reaction feasibility based on multiple tasks and molecular multi-modal features, comprising:
a data obtaining module configured to supplement negative samples for a public feasible enzymatic reaction dataset and convert the public feasible enzymatic reaction dataset into a dataset in the form of molecule pairs in combination with labels as needed by a model, wherein the molecule pair is composed of a single substrate molecule SMILES and a single product molecule SMILES;
a molecular multi-modal feature extraction module configured to extract multi-modal features of molecules according to the dataset in the form of the molecule pairs in combination with the labels, wherein the multi-modal features comprise a molecular sequence feature and a structure feature;
an enzymatic reaction feasibility evaluation module configured to input the multi-modal features of a molecule pair to a deep learning model, allow the model to further extract and associate features, and output a feasibility evaluation value of a corresponding reaction; and
a multi-task driven model performance optimization module configured to combine an output of the model with a multi-task calculation error loss for feeding back to the model for further parameter optimization, wherein multiple tasks comprise a product SMILES sequence generation task and a binary classification task for reaction feasibility.
